(19) United States
(12) Patent Application Publication
(10) Pub. No.: US 2019/0333624 A1
(43) Pub. Date: Oct. 31, 2019

(54) MEAL PLANNING APPARATUSES, SYSTEMS AND METHODS WITH SUPPLY-DEMAND COORDINATION

(71) Applicant: Xuan Huang, Birmingham, AL (US)

(72) Inventor: Xuan Huang, Birmingham, AL (US)

(21) Appl. No.: 16/394,640

(22) Filed: Apr. 25, 2019

Related U.S. Application Data
(60) Provisional application No. 62/663,230, filed on Apr. 26, 2018.

(51) Int. Cl. G16H 20/60; G06N 7/00; G06F 17/18

(52) U.S. Cl. CPC G16H 20/60; G06F 17/18; G06N 7/005

(57) ABSTRACT

Disclosed herein is a server computer comprising a processor configured to adjust one or more variables in meal plans respectively for a plurality of consumers under a first set of constraints comprising a constraint on collective supply of food items of the meal plans and constraints on respective demand by the plurality of consumers for the food items of the meal plans, until a termination condition is satisfied. Also disclosed are a system including the server computer and a method of meal planning.

### CROSS-REFERENCE TO RELATED APPLICATION

This application claims priority to U.S. Provisional Patent Application No. 62/663,230, filed on Apr. 26, 2018, the contents of which are incorporated by reference in the entirety.

### BACKGROUND

A meal occurs multiple times a day at various locations for most people. Special meals may occur at special events, such as gatherings for birthdays, weddings, various holidays for cultural, religious, and personal reasons. On some occasions, a meal may include several courses including drinks and desserts; on other occasions it may include light serving of snacks and fruits. Planning for a meal often uses knowledge and research on what the consumer for the meal should or likes to consume, the amount it would consume, and how to prepare the dishes that constitute the meal.

For most families, planning, shopping for, and preparing for meals is time consuming and may lead to waste of food. Waste does not only happen at a household. Natural Resources Defense Council estimates 40% of food in America is wasted across the food supply chain, from farm to fork to landfill, including spoilage at the supermarkets. Another report by the United States Department of Agriculture (USDA) found that annual supermarket losses for 2005 and 2006 averaged 11.4% for fresh fruit, 9.7% for fresh vegetables, and 4.5% for fresh meat, poultry, and seafood. Supermarkets often run promotions in anticipation of oversupply of certain food items, but it does not eliminate the mismatch between supply and demand before the food items pass their prime time.

With all the challenges outlined above, a better method is needed to make it more enjoyable to prepare meals, to make meals more nutritious and personalized, to reduce waste across the food supply chain, and to make it easy to track food consumption data at the individual level.

### SUMMARY

Disclosed herein is a server computer comprising a processor configured to adjust one or more variables in meal plans respectively for a plurality of consumers under a first set of constraints comprising a constraint on collective supply of food items of the meal plans and constraints on respective demand by the plurality of consumers for the food items of the meal plans, until a termination condition is satisfied.

According to an embodiment, the termination condition is selected from a group consisting of minimization of an objective function, maximization of the objective function, a certain number of iterations reached, a value of the objective function being equal to or beyond a certain threshold value, a certain computation time reached, a value of the objective function being within an acceptable error limit, and combinations thereof.

According to an embodiment, the objective function represents an amount of waste of the food items.

According to an embodiment, the objective function representing the amount of waste of the food items is a function of a discrepancy between supply and demand of at least one of the food items.

According to an embodiment, the processor is configured to adjust the one or more variables under a second set of constraints including a constraint the consumers impose after reviewing the meal plans, a constraint imposed by a change of projected or actual supply of the food items, or a combination thereof.

According to an embodiment, the processor is configured to adjust the one or more variables under a third set of constraints that set at least one of the variables to a specific value.

According to an embodiment, the processor is configured to estimate the collective supply using estimating the collective supply, a Holt-Winters model, an autoregressive integrated moving average (ARIMA) model, or a combination thereof.

According to an embodiment, the variables in the meal plan comprise quantities of at least some of the food items.

According to an embodiment, the meal plan comprises recipes, descriptions of food items, nutritional facts of the food items, order intervals or a combination thereof.

According to an embodiment, the constraint the consumers impose comprises acceptance of the meal plan, rejection of the meal plan, setting values of one or more of the variables, or combinations thereof.

According to an embodiment, at least one variable of the meal plans is a discrete variable.

According to an embodiment, the first set of constraints comprises constraints on nutrients of the meal plans, certain characteristics of the meal plans required by the consumers, locations, events, groups of the consumer, locations and events, the consumers' preference scores, amounts of the food items in the consumers' possession, allergies of the consumers, budgets of the consumers, or combinations thereof.

Disclosed herein is a system comprising: the above server computer described above, a storage, and a communication interface, wherein the communication interface is configured to receive the first set of constraints from the storage, from a first remote computer, or from both, and configured to provide the first set of constraints to the processor.

According to an embodiment, the communication interface is configured to transmit the meal plan to a second remote computer.

According to an embodiment, the storage is configured to provide the first set of constraints stored therein to the communication interface at a request of the processor.

According to an embodiment, the communication interface comprises a gateway, a router, a network bridge, a modem, a wireless access point, a switch, a hub, a repeater, a proxy server, a firewall, a network address translator, a multiplexer, a network interface controller, a wireless network interface controller, or a combination thereof.

Disclosed herein is a method, comprising: adjusting, using a processor of a server computer, one or more variables in meal plans respectively for a plurality of consumers under a first set of constraints comprising a constraint on collective supply of food items of the meal plans and constraints on respective demand by the plurality of consumers for the food items of the meal plans, until a termination condition is satisfied.

According to an embodiment, the termination condition is selected from a group consisting of minimization of an objective function, maximization of the objective function, a certain number of iterations reached, a value of the objective function being equal to or beyond a certain threshold value, a certain computation time reached, a value of the objective function being within an acceptable error limit, and combinations thereof.

According to an embodiment, the objective function represents an amount of waste of the food items.

According to an embodiment, the objective function representing the amount of waste of the food items is a function of a discrepancy between supply and demand of at least one of the food items.

According to an embodiment, adjusting the one or more variables is under a second set of constraints including a constraint the consumers impose after reviewing the meal plans, a constraint imposed by a change of projected or actual supply of the food items, or a combination thereof.

According to an embodiment, adjusting the one or more variables is under a third set of constraints that set at least one of the variables to a specific value.

According to an embodiment, the method further comprises estimating the collective supply using estimating the collective supply, a Holt-Winters model, an autoregressive integrated moving average (ARIMA) model, a heuristic algorithm, a k-means clustering algorithm, or a combination thereof.

According to an embodiment, the variables in the meal plan comprise quantities of at least some of the food items.

According to an embodiment, the meal plan comprises recipes, descriptions of food items, nutritional facts of the food items, order intervals or a combination thereof.

According to an embodiment, the constraint the consumers impose comprises acceptance of the meal plan, rejection of the meal plan, setting values of one or more of the variables, or combinations thereof.

According to an embodiment, the method further comprises repeating adjusting the one or more variables when the second set of constraints is changed.

According to an embodiment, the method further comprises updating the amounts of the food items in the consumers' possession by collecting information from packing materials discarded by the consumers.

According to an embodiment, the method further comprises transmitting the meal plan to a supplier of the food items.

According to an embodiment, the first set of constraints comprises constraints on nutrients of the meal plans, certain characteristics of the meal plans required by the consumers, locations, events, groups of the consumer, locations and events, the consumers' preference scores, amounts of the food items in the consumers' possession, allergies of the consumers, budgets of the consumers, or combinations thereof.

Disclosed herein is a computer program product comprising a non-transitory computer readable medium having instructions recorded thereon, the instructions when executed by a computer implementing any of the above methods.

### BRIEF DESCRIPTION OF FIGURES

FIG. 1 schematically shows a system for meal planning, in particular meal planning through optimization under constraints on both supply and demand of food items of the meal plan, according to an embodiment.

FIG. 2A-FIG. 2D each schematically show a partial functional block diagram for the system for the meal planning, according to an embodiment.

### DETAILED DESCRIPTION

Food items are unique products in that the demand is highly flexible, and there are many arrangements that may satisfy a consumer's need. For example, a consumer who initially wanted mashed potatoes may find mashed sweet potatoes equally desirable. Salsa of one brand can be substituted with that from a different brand. Quality of grocery does matter, however demands can be influenced by curated choices.

The supply of food items, however, is less flexible, at least in the short term. Each food item goes through its own biological and/or manufacturing cycle. It is relatively difficult to shorten or prolong that cycle. Nevertheless, a supplier for food items may make a reasonable projection on the availability of each food item and its amount for the near future. The projected availability enables planning meals ahead by a reasonable time window. This creates a unique opportunity for the food item supplier, a distributor, or a third party vendor or planner, to coordinate, optimize, and recommend meal plans to a consumer according to the known or projected availability of the food items, personal taste of the consumer, nutrition goals of the consumer, dietary restrictions of the consumer, meal occasions, budgets of the consumer, and the consumer's own inventory. The term “consumer” as used herein may mean a single person or a group of persons (e.g., members of a family, participants of a special event, students in a school, persons grouped together using a clustering algorithm such as the k-means clustering algorithm, individuals who share certain characteristics such as health conditions, religions, geographic locations) who uses a meal plan. A plurality of persons may be grouped into a plurality of consumers using a suitable clustering algorithm. A consumer is not necessarily a person or group of persons who actually ingests the food items in the meal plan, but may be a food processing entity such as a cafeteria in a school or a restaurant.

A meal plan comprises a set of variables selected from a group consisting of quantities of food items, numbers of servings of one or more recipes, processing parameters of food items, and combinations thereof. The meal plan may include information such as time of delivery, nutritional facts of the meal plan, costs of the meal plan, types of cuisines, types of diets, difficulty levels of preparing meals from the food items, time needed of preparing meals from the food items, origins of the food items, carbon footprints of the food items, or a combination thereof. According to an embodiment, examples of food items may include fresh produce, processed food, meat, fish, poultry, dairy products, spices, and raw ingredients such as flour, sugar, cooking oil, partially or fully cooked meals, wines, liquors, beers, and other drinks.

Some variables in a meal plan may be discrete variables. For example, the quantity of certain food items may be a discrete variable because of the packaging of the food item. A food item in a meal plan may not be portioned for depletion within a certain time frame. Instead, some of the food item may be used at once and a remainder of the food item may be kept in a consumer's inventory. For example, a bottle of vinegar may be partially consumed during one meal and gradually depleted over a long time. The remainder of the food item in the consumer's inventory may be tracked.

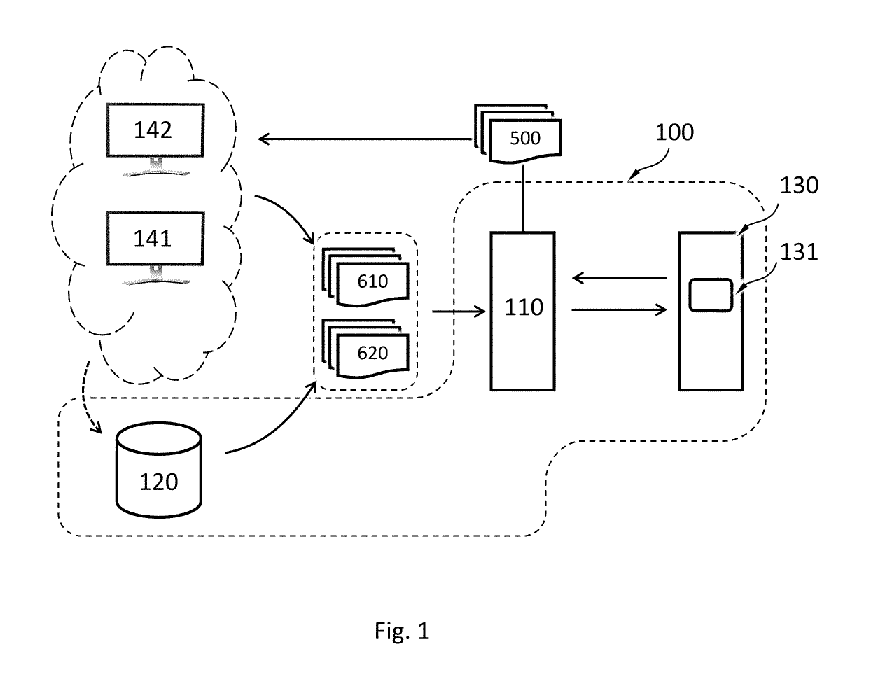

FIG. 1 schematically shows a system 100 for meal planning, according to an embodiment. The system 100 includes a communication interface 110, a storage 120, and a server computer 130 having a processor 131. The communication interface 110 is a hardware component configured to receive a first set 610 of constraints from the storage 120, from a first remote computer 141, or from both, and is configured to provide the first set 610 of constraints to the processor 131 of the server computer 130. The communication interface 110 may communicate with the first remote computer 141 or the storage 120 through a network (e.g. the Internet). Examples of the communication interface 110 may include a gateway, a router, a network bridge, a modem, a wireless access point, a switch, a hub, and a repeater, a proxy server, a firewall, a network address translator, a multiplexer, a network interface controller, a wireless network interface controller, or a combination thereof.

The communication interface 110 may also be configured to transmit other data between the processor 131 of the server computer 130 and the storage 120, between the processor 131 of the server computer 130 and the first remote computer 141, or between the processor 131 of the server computer 130 and a second remote computer 142. The first remote computer 141 and the second remote computer 142 may be the same or different computers. Examples of the first remote computer 141 and the second remote computer 142 may include smart phones, tablets, game consoles, desktop computers, laptop computers and other suitable devices.

The storage 120 may include storage media such as a hard drive, a solid-state drive, a memory or other suitable hardware that can store information. The information may be organized in any suitable fashion, for example, as a database. The storage 120 have various information relevant to meal planning stored therein. The information may include recipes, consumer information, consumers' inventories of food items, nutritional information and allergen information, diet regimens, and dietary guidelines, consumer meal plan histories and feedback, or a combination thereof.

The recipes stored in the storage 120 may include ingredients, preparing instructions, equipment needed, nutritional facts, information on allergens, diet types (e.g., vegan, paleo, etc.), types of meal (e.g., appetizers, salad, soup, lunch, snack, drink, etc.), ethnic origins, culture significance (e.g., turkey for Thanksgiving, cakes for birthdays, etc.), average rating, difficulty levels, time needed for preparing, flavor profile (e.g., savory, sweet, umami, etc.), costs, lists of similar or substitute recipes, trendiness, and pictures, videos, or comments of food items. The recipes stored in the storage 120 may be grouped as a set of active recipes and a set of off-season recipes or rotation-off recipes.

The consumer information stored in the storage 120 may include consumers' demographics, body measurements, health histories or concerns (e.g., weight-loss, muscle-building, pregnancy, etc.), diet preferences (e.g., keto, etc.), dietary restrictions or allergies (vegan, gluten-free, etc.), flavor preferences, willingness of trying new types of food, difficulty level preferences, budgets, and subscription information (e.g., delivery schedule, meal plan inclusions). Some of the consumer information may be received from the first remote computer 141, as shown in FIG. 1. Some of the consumer information may be derived implicitly from other information stored in the storage 120. In one embodiment, the consumer information may be inferred through acceptance of meal plans by consumers, rejection of meal plans by consumers, changes to meal plans by consumers, their meal plan histories and their feedback, or combinations thereof.

The consumers' inventories of food items may include amounts of food items consumers possess, and additional information such as the consumers' histories of inventories of the food items, information on scheduled delivery of the food items to the consumers, or a combination thereof.

The diet regimens and dietary guidelines may include up-to-date data derived from USDA dietary guidelines, various diet regimens, or may be created by health professionals and customized for their patients. In an embodiment, the recommended upper limit for calories from added sugars is 10% per day, according to the USDA dietary guidelines 2015-2020. In an embodiment, a primary care doctor may customize a meal plan to meet his pregnant patient's dietary requirements that more than 600 mcg folic acid is recommended for her second trimester and onward.

The consumer histories may include records of every meal consumers ordered and related ratings and feedbacks of the meal.

According to an embodiment, the storage 120 is configured to provide information stored therein (e.g., the first set 610 of constraints) to the communication interface 110 at a request of the processor 131.

According to an embodiment, the processor 131 of the server computer 130 is configured to (i.e., specially programmed to) adjust one or more variables in meal plans 500 respectively for a plurality of consumers under the first set 610 of constraints until a termination condition is satisfied. The first set 610 of constraints comprises a constraint on the collective supply of the food items of the meal plans 500 and constraints on the respective demand by the plurality of consumers for these food items. The collective supply and the demand may be actually known or may be estimated based on other information. In an embodiment, the termination condition is selected from a group consisting of minimization of an objective function, maximization of the objective function, a certain number of iterations reached, a value of the objective function being equal to or beyond a certain threshold value, a certain computation time reached, a value of the objective function being within an acceptable error limit, and combinations thereof. In an embodiment, the objective function represents an amount of waste of the food items of the meal plans 500. In an embodiment, the objective function is a function of a discrepancy between the supply and demand of at least one of the food items of the meal plans 500. In an embodiment, the objective function represents the dollar value of the total amounts of waste of the food items of the meal plans 500. In another embodiment, the objective function represents the sum of all consumers' preference scores. In yet another embodiment, the objective function represents a weighted combination of several goals including nutrition, preference, and waste.

Throughout this disclosure, the following mathematical notations are used:

T: the time point at which the food items included in the meal plans have to be available and fulfilled;
c∈{1, 2, . . . C}: index for consumer; C is the total number of consumers being considered collectively;
r∈{1, 2, . . . R}: index for recipe; R is the total number of recipes;
i∈{1, 2, . . . I}: index for food item; I is the total number of food items;
n∈{1, 2, . . . N}: index for nutrient; N is the total number of nutrients;
m∈{1, 2, . . . m_{eT}}: index for meal (e.g., in one embodiment, each customer consumes 4 meals a day: m=1—breakfast, m=2—lunch, m=3—dinner, m=4—snack, m=5—breakfast, m=6—lunch, . . . etc. In other words, m %4 (m mode 4)=1—breakfast, m %4=2—lunch, m %4=3—dinner, and m %4=0—snack);
m_{cT}: a parameter to denote the total number of meals needed to be planned for consumer cat time T;
aᵣᵢ: a parameter to denote the amount of food item i called for by recipe r;
bᵣₙ: a parameter to denote the amount of nutrient n contained by recipe r;
c_{rT} a parameter to denote the cost for one serving of recipe rat time T;
p_{crT} a parameter to denote consumer c's preference score to recipe rat time T;
S_{iT}: a parameter to denote supply for food item i at time T; S_{iT} may be projected (e.g., through the optimizer 132) or actual and its value may be updated from time to time;
d_{cm}: a parameter to denote the number of servings of recipes of consumer cat meal m; it may be inferred through the optimizer 132;
y_{cmrT} a decision variable to denote the number of servings of recipe r will be consumed by consumer cat meal m;
σ_{iT}: a slack variable to denote remaining/surplus/wasted amount of food item i at time T. There may be other parameter to represent information stored at storage 120, for example, vᵣ=1 if recipe r is vegetable dish and 0 otherwise.

The processor 131 of the server computer 130 may be configured to adjust the one or more variables of the meal plans 500 under a second set 620 of constraints. The second set 620 of constraints includes a constraint the consumers impose after reviewing the meal plans 500, a constraint imposed by a change of projected or actual supply of the food items of the meal plans 500, additional, relaxation of, or tightening of constraints on nutrients of the meal plans 500, recipe preference by a consumer, amounts of the food items in a consumer's possession, allergies of a consumer, budgets of a consumer, relationship to a second consumer, certain characteristics of meal plan required by consumer, or combinations thereof.

The processor 131 of the server computer 130 may be configured to adjust the one or more variables of the meal plans 500 under a third set of constraints that set at least one of the variables to a specific value. Namely, the values of some of the variables may be fixed.

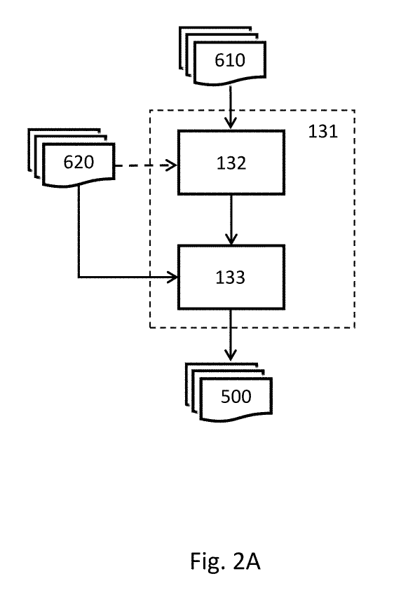

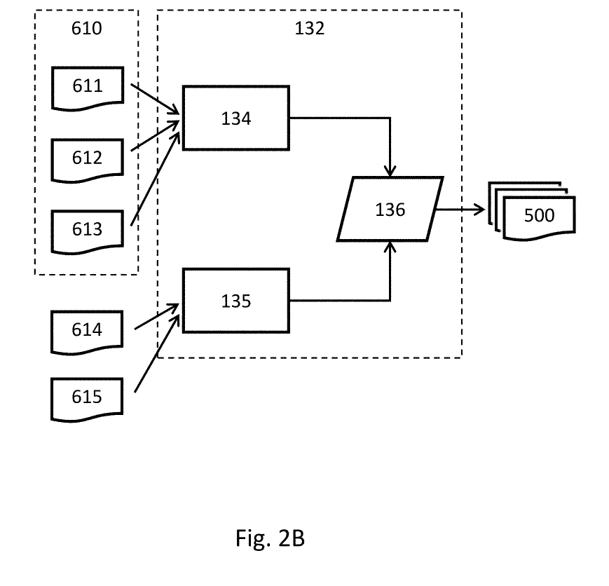

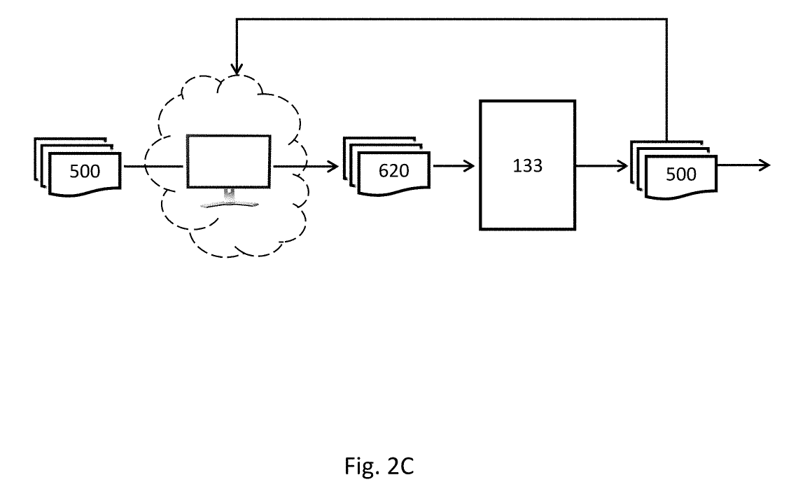

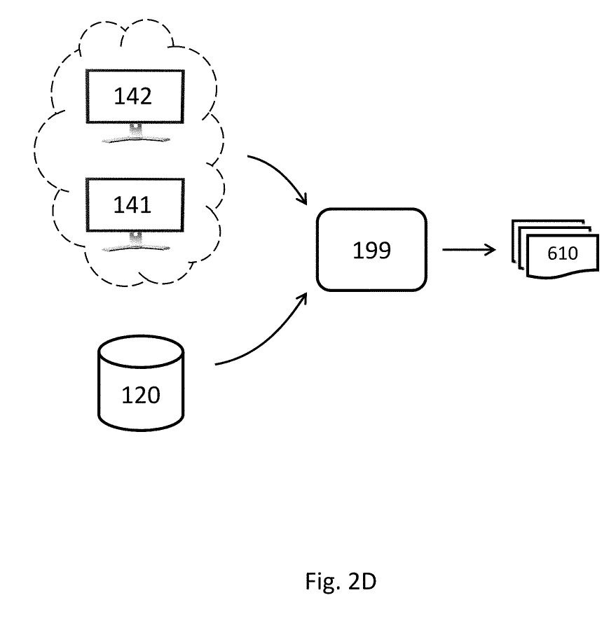

FIG. 2A schematically shows a partial functional block diagram of the processor 131, according to an embodiment. The processor 131 may include an optimizer 132, and an optimizer 133. The optimizer 132 may adjust (e.g., set the initial values of) the one or more variables of the meal plan 500 under the first set 610 of constraints on the supply and demand of the food items, until the termination condition is satisfied. The optimizer 133 may further adjust the variables under the second set 620 of constraints. The processor 131 may receive the second set of 620 of constraints and adjust the variables iteratively. For example, after the optimizer 133 further adjusts the variables, the meal plan 500 may be presented to the consumer and consumer may alter the second set 620 of constraints, and the optimizer 133 then further adjusts the variables under the altered second set 620 of constraints. FIG. 2B schematically shows a partial functional diagram of the optimizer 132, according to an embodiment. The optimizer 132 may receive the first set 610 of constraints on the supply and demand from the storage 120, or from the first remote computer 141, or from both, via the communication interface 110. In an embodiment, the first set 610 of constraints includes a constraint 611 imposed by the consumer's order history, which is a constraint on the demand. The constraint 611 may limit the amount of deviation from the meal plans in the consumer's order history. In an embodiment, the first set 610 of constraints includes a constraint 612 imposed by delivery schedules of the consumer (i.e., the consumer's subscription), which is a constraint on the demand. For example, for a consumer c who need 4 meals a day and is on a weekly plan with deliveries on Mondays and Thursdays, the delivery for Monday (T=2) would be m_{cr}=3 days×4 meals/day=12 meals and the delivery for Thursday (T=5) would be m_{cT}=4 days×4 meals/day=16 meals. For example, for another consumer c who is on a two-day plan, every delivery would be m_{cT}=2 days×4 meals/day=8 meals. The optimizer 132 may have an estimator 134 programmed to estimate the demand of food items of a meal m for a consumer c based on the order history constraints and subscription of the consumer c. In an embodiment, the demand of the food items is a function of the number of servings in the meal plan. The total number of servings may be constrained to equal d_{cm}:

$${\sum }_{1 \leq r \leq R} {y}_{cmrT} = {d}_{c m} , ∀ c , m .$$

For example, if a consumer c may require that at the time T, he or she wants one and only one serving for meal m, d_{cm} is then fixed to the value of 1.

In an embodiment, the first set 610 of constraints includes a constraint 613 imposed by the total available supply of the food items. For example, the food items at the time T may be limited and the total demand of the food items should not exceed their supply. The constraint 613 may include variables y_{cmrT} or their aggregation x_{rT}, and the supply of food items S_{iT}. In an example, the relationship between variables y_{cmrT} and their aggregation x_{rT}, and the relationship between x_{rT} and S_{iT} may be expressed as:

$${\sum }_{1 \leq c \leq C} {\sum }_{1 \leq m \leq {m}_{cT}} {y}_{cmrT} = {x}_{rT} , ∀ r$$

$${\sum }_{1 \leq r \leq R} {x}_{rT} {a}_{ri} + {σ}_{iT} = {s}_{iT} , ∀ i$$

wherein aᵣᵢ is amount of food item i of recipe r, x_{rT} is the total servings of recipe r for all the consumers c, S_{iT} is the actual or projected supply of food item i, σ_{iT} is discrepancy between the supply and demand of food item i at time T. One example of the constraint may be that σ_{iT}≥0, i.e., the supply of food items S_{iT} must be sufficient to satisfy the collective demand.

The supply of the food items may be estimated in any suitable way. In an embodiment, the optimizer 132 has an estimator 135. The estimator 135 may estimate the supply based on information 614 received from the storage 120, e.g., suppliers' inventories, a scheduled delivery amount and date, on a manually entered empirical guess 615, or on a combination thereof. The estimator 135 may use a seasonally adjusted moving average, a Holt-Winters model, or an ARIMA (Autoregressive integrated moving average) model to estimate the supply. The restocking of the food items may be temporally smoothed. In an embodiment, oranges are restocked in a first week for 140 cases, and another 70 cases in a second week. The restock may be smoothed out, for example, to restock 20 cases every day for the first week, and restock 10 cases every day for the second week, or restock 15 cases every day for both weeks. Smoothing algorithms for the supply may take into account particular food items storage shelf life and ensure the food items may be delivered at prime times.

A function 136 may programmed to determine meal plans 500 for the consumers, based on the demand estimate of food items d_{cm} from the estimator 134 and the supply estimate food items S_{iT} from the estimator 135, as shown in FIG. 2B. The function 136 may repeatedly adjust multiple variables in the meal plan to satisfy the termination condition.

FIG. 2C schematically shows that the meal plans 500 may be transmitted to the remote computer (e.g. 141 or 142 in FIG. 2A) via the communication interface 110 for the consumers of the meals plans or for the suppliers of the food items to review the meal plans 500, according to an embodiment. The consumers or suppliers may provide some or all of the constraints in the second set 620 of constraints, which may include a constraint the consumers impose after reviewing the meal plans 500, a constraint imposed by a change of projected or actual supply of the food items of the meal plans 500, constraints on nutrients of the meal plans 500, additional, relaxation of, or tightening of constraints on nutrients of the meal plans 500, recipe preference by a consumer, amounts of the food items in a consumer's possession, allergies of a consumer, budgets of a consumer, relationship to a second consumer, certain characteristics of meal plan required by consumer, groups of the consumer, locations and events, or combinations thereof. The second set 620 of constraints may include some or all constraints of the first set 610. The optimizer 133 further adjusts the variables of the meal plans 500 under the second set 620 of constraints. The meal plans 500 produced by the optimizer 133 may be transmitted again to the remote computer (e.g. 141 or 142 in FIG. 2A) via the communication interface 110 for the consumers of the meals plans or for the suppliers of the food items to further review the meal plans 500. In an example, where the meal plan 500 for a certain consumer c₁ is presented to that consumer and that consumer decides certain recipe r₁ in the meal plan 500 is not desirable, the second set 620 of constraints will then include a constraint y_{c}_{₁}ₘᵣ_{₁}_{T}=0, ∀m. In an example, where the meal plans 500 are produced by the optimizer 133 under the condition that the supply of food item i is S_{iT}, and the supplier for food item i later finds out that the supply of food item i is S_{iT}′ instead of S_{iT}, the second set 620 of constraints will then impose a new constraint based on the supply S_{iT}′.

In an embodiment, the constraint the consumers impose after reviewing the meal plans 500 includes acceptance of the meal plans 500, rejection of the meal plans 500, or setting values of one or more of the variables, or combinations thereof. When the consumer accepts the meal plans 500, the optimizer 133 may finalize the meal plans 500 and transmit the meal plans 500 to the communication interface 110 to fulfil orders. When the consumer rejects the meal plans 500, the optimizer 133 may adjust one or more variables under the second set 620 of constraints provided by the consumer.

The first set 610 of constraints may include a nutrients constraint for consumer c, as expressed below,

R_{c}^{N}(Σ_{1≤r≤R}Y_{cmrt}bᵣₙ), where t≤T,1≤m≤m_{ct},1≤n≤N

wherein R_{c}^{N}(·) may be a set of constraints that describe consumer c's nutritional needs. It may be a dietary guideline, for example, protein consumption at at least 0.8 grams of protein per kilogram of body weight, or it may be a diet regime such as ratios between protein, fat, and carbs may be approximately 1:1:2 for each meal, or added sugar consumption may be less than 20 g a day. The constraints may be imposed on one meal, or may be over a time span (may include a certain span of the history of t≤T). R_{c}^{N}(·) may be nonlinear constraints. The nutrients constraints may be simplified as a health scores assigned to each recipe, and therefore to the consumer's meal plans. For example, Σ_{1≤r≤R} y_{cmrT}sᵣ≥H_{c}, ∀c, m where Sᵣ is the health score for recipe r and H_{c} is the health threshold for consumer c.

The first set 610 of constraints may include a budget constraint for group g at meal m, as expressed below,

$${R}_{gmT}^{B} ( {\sum }_{c ∈ \{ c \vert {g}_{cmT} = g \}} {\sum }_{1 \leq r \leq R} {y}_{cmrt} {c}_{rT} )$$

customers may belong to a long-term group (e.g., a family), or a short-term group, (e.g., a birthday party dinner enjoyed by family and friends). g_{cmT} may denote a group number for customer c at meal m. R_{gmT}^{B}(·) may be a budget constraint for the group g at a certain meal m. Budget constraints may be restricting the cost of one meal or several meals for the group to be within certain limits. R_{gmT}^{B}(·) may be a linear constraint, e.g., Σ_{c∈{c|g}_{_{cmT}}_{=g}}Σ_{1≤r≤R}y_{cmrt}c_{rT}≤B_{gmT},∀g,m wherein B_{gmT}, is the budget for group g at meal m.

The first set 610 of constraints may include a preference constraint, as expressed below

R_{c}^{P}(Σ_{1≤r≤R}y_{cmrT}p_{crT})

R_{c}^{P}(·) may be a preference constraint for customer c. p_{crT} may be a preference parameter for customer c for recipe r at time T. For example, p_{crT} may be the consumer's previous rating for one particular recipe. When the recipe is new to the consumer, p_{crT} may be inferred from the consumer's rating on similar recipes that are stored in the storage 120 or similar consumers' ratings on this recipe that are stored in the storage 120. This parameter may be adaptive over time as more data being collected. When the consumer prefers a varied diet, the preference parameter may be adjusted accordingly for novel recipes that the customer may have not tried before. In an embodiment, the preference constraint may be taking food pairing and wine pairing into account. R_{c}^{P}(·) may be linear constraints, for example, Σ_{1≤r≤R}y_{cmrT}p_{crT}≥P_{c}, V_{c, m} wherein P_{c} is the preference threshold for consumer c.

The first set 610 of constraints may include an allergen constraint, as expressed below,

$${\sum }_{1 \leq r \leq R} {y}_{cmrt} {a}_{ri} = 0 ∀ i ∈ \{ i \vert {A}_{ci} = 1 \}$$

A_{ci}=1 if food item i may contain allergens for consumer c or equals 0 otherwise.

The first set 610 of constraints may include a constraint of consumer group. In an embodiment, customer c₁ and c₂ are spouses, and they consume the same portion of recipes together at any time point, therefore constraints can be constructed such that y_{c}_{₁}_{mrT}=y_{c}_{₂}ₘᵣₜ wherein g_{c}_{₁}_{mT}=g_{c}_{₂}_{mT}·g_{c}_{₁}_{mT} is the group number for consumer c₁ at meal m. The consumer group constraint may be designed to accommodate different group members requiring different portion of servings and certain group member to have more specific restrictions. Alternatively, a family may choose to be divided into different groups. For example, a family with a pregnant mom may choose to separate her meal plan with the rest of the family as her diet may require higher standards for nutrition.

The first set 610 of constraints may include a location and event constraint. Each recipe may also have tags to indicate certain property of the recipe. In an embodiment, dᵣ=1 means recipe r is easy to pack. If a consumer always brings lunch to work, he may choose his location constraints to be dᵣ=1, ∀m %4=2 & r such that y_{cmrT}=1. Similar restrictions may be created for Thanksgiving meals, birthday meals, etc., to ensure the meal plan may fit for the events or locations. The first set 610 may include other constraints on the demand. For example, a constraint imposed by a consumer's requirement that every meal contains at least one serving of vegetable may be expressed as

$${\sum }_{1 \leq r \leq R} {y}_{cmrT} {v}_{r} \geq 1 , ∀ c , m$$

where vᵣ=1 if recipe r is vegetable dish and 0 otherwise.

FIG. 2D schematically shows that some of the first set 610 of constraints may be generated. In an embodiment, a processor 199, which may be the same as or different from the processor 131, may generate some of the first set 610 of constraints by determining values of some parameters of these constraints based on information from a remote computer (e.g., remote computer 141 or 142) or the storage 120. In an example, S_{iT} may be projected based on information such as a contracted delivery amount and date, a manually entered expert guess, a simple seasonally adjusted moving average, a Holt-Winters model, or through a time series model such as the ARIMA model. In another embodiment, the optimization can be carried out as a two-stage problem: at the first stage, recipes and their servings will be determined according to the (projected) supply; at the second stage, recipes will be assigned to consumers according to their individual nutrition, budget, preference constraints.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A server computer comprising a processor configured to adjust one or more variables in meal plans respectively for a plurality of consumers under a first set of constraints comprising a constraint on collective supply of food items of the meal plans and constraints on respective demand by the plurality of consumers for the food items of the meal plans, until a termination condition is satisfied.

2. The server computer of claim 1, wherein the termination condition is selected from a group consisting of minimization of an objective function, maximization of the objective function, a certain number of iterations reached, a value of the objective function being equal to or beyond a certain threshold value, a certain computation time reached, a value of the objective function being within an acceptable error limit, and combinations thereof.

3. The server computer of claim 2, wherein the objective function represents an amount of waste of the food items.

4. The server computer of claim 3, wherein the objective function representing the amount of waste of the food items is a function of a discrepancy between supply and demand of at least one of the food items.

5. The server computer of claim 1, wherein the processor is configured to adjust the one or more variables under a second set of constraints including a constraint the consumers impose after reviewing the meal plans, a constraint imposed by a change of projected or actual supply of the food items, or a combination thereof.

6. The server computer of claim 1, wherein the processor is configured to adjust the one or more variables under a third set of constraints that set at least one of the variables to a specific value.

7. The server computer of claim 1, wherein the processor is configured to estimate the collective supply using a seasonally adjusted moving average, a Holt-Winters model, an autoregressive integrated moving average (ARIMA) model, or a combination thereof.

8. The server computer of claim 1, wherein the variables in the meal plan comprise quantities of at least some of the food items.

9. The server computer of claim 1, wherein the meal plan comprises recipes, descriptions of food items, nutritional facts of the food items, order intervals or a combination thereof.

10. The server computer of claim 5, wherein the constraint the consumers impose comprises acceptance of the meal plan, rejection of the meal plan, setting values of one or more of the variables, or combinations thereof.

11. The server computer of claim 1, wherein at least one variable of the meal plans is a discrete variable.

12. The server computer of claim 1, wherein the first set of constraints comprises constraints on nutrients of the meal plans, certain characteristics of the meal plans required by the consumers, locations, events, groups of the consumer, locations and events, the consumers' preference scores, amounts of the food items in the consumers' possession, allergies of the consumers, budgets of the consumers, or combinations thereof.

13. A system comprising:
the server computer of claim 1,
a storage, and
a communication interface,
wherein the communication interface is configured to receive the first set of constraints from the storage, from a first remote computer, or from both, and configured to provide the first set of constraints to the processor.

14. The system of claim 12, wherein the communication interface is configured to transmit the meal plan to a second remote computer.

15. The system of claim 12, wherein the storage is configured to provide the first set of constraints stored therein to the communication interface at a request of the processor.

16. The system of claim 12, wherein the communication interface comprises a gateway, a router, a network bridge, a modem, a wireless access point, a switch, a hub, a repeater, a proxy server, a firewall, a network address translator, a multiplexer, a network interface controller, a wireless network interface controller, or a combination thereof.

17. A method, comprising:
adjusting, using a processor of a server computer, one or more variables in meal plans respectively for a plurality of consumers under a first set of constraints comprising a constraint on collective supply of food items of the meal plans and constraints on respective demand by the plurality of consumers for the food items of the meal plans, until a termination condition is satisfied.

18. The method of claim 17, wherein the termination condition is selected from a group consisting of minimization of an objective function, maximization of the objective function, a certain number of iterations reached, a value of the objective function being equal to or beyond a certain threshold value, a certain computation time reached, a value of the objective function being within an acceptable error limit, and combinations thereof.

19. The method of claim 18, wherein the objective function represents an amount of waste of the food items.

20. The method of claim 19, wherein the objective function representing the amount of waste of the food items is a function of a discrepancy between supply and demand of at least one of the food items.

21. The method of claim 17, wherein adjusting the one or more variables is under a second set of constraints including a constraint the consumers impose after reviewing the meal plans, a constraint imposed by a change of projected or actual supply of the food items, or a combination thereof.

22. The method of claim 17, wherein adjusting the one or more variables is under a third set of constraints that set at least one of the variables to a specific value.

23. The method of claim 17, further comprising estimating the collective supply using a seasonally adjusted moving average, a Holt-Winters model, an autoregressive integrated moving average (ARIMA) model, or a combination thereof.

24. The method of claim 17, wherein the variables in the meal plan comprise quantities of at least some of the food items.

25. The method of claim 17, wherein the meal plan comprises recipes, descriptions of food items, nutritional facts of the food items, order intervals or a combination thereof.

26. The method of claim 21, wherein the constraint the consumers impose comprises acceptance of the meal plan, rejection of the meal plan, setting values of one or more of the variables, or combinations thereof.

27. The method of claim 21, further comprising repeating adjusting the one or more variables when the second set of constraints is changed.

28. The method of claim 17, further comprising updating the amounts of the food items in the consumers' possession by collecting information from packing materials discarded by the consumers.

29. The method of claim 17, further comprising transmitting the meal plan to a supplier of the food items.

30. The method of claim 17, wherein the first set of constraints comprises constraints on nutrients of the meal plans, certain characteristics of the meal plans required by the consumers, locations, events, groups of the consumer, locations and events, the consumers' preference scores, amounts of the food items in the consumers' possession, allergies of the consumers, budgets of the consumers, or combinations thereof.

31. A computer program product comprising a non-transitory computer readable medium having instructions recorded thereon, the instructions when executed by a computer implementing a method of claim 17.
